(19) United States
(12) Patent Application Publication
(10) Pub. No.: US 2022/0023335 A1
(43) Pub. Date: Jan. 27, 2022

(54) COMPOSITION FOR PREVENTION AND/OR IMPROVEMENT OF ADVERSE DRUG REACTION, SYMPTOM ASSOCIATED WITH ADVERSE DRUG REACTION, AND/OR ADVERSE REACTION ASSOCIATED WITH MEDICAL TREATMENT

(71) Applicant: MiZ Company Limited, Kanagawa (JP)

(72) Inventors: Fumitake SATOH, Kanagawa (JP); Bunpei SATO, Kanagawa (JP); Yusuke ICHIKAWA, Kanagawa (JP); Shinichi HIRANO, Kanagawa (JP); Yoshihiro MITEKURA, Kanagawa (JP)

(73) Assignee: MiZ Company Limited, Kanagawa (JP)

(21) Appl. No.: 17/376,754

(22) Filed: Jul. 15, 2021

(30) Foreign Application Priority Data
Jul. 21, 2020 (JP) 2020-136852

(51) Int. Cl. A61K 33/00; A61P 39/00

(52) U.S. Cl. CPC A61K 33/00; A61P 39/00

(57) ABSTRACT

The present invention provides a composition for prevention and/or improvement of an adverse drug reaction, a symptom associated with an adverse drug reaction, and/or an adverse reaction associated with medical treatment. More specifically, the present invention provides a composition for prevention and/or improvement of an adverse drug reaction, a symptom associated with an adverse drug reaction, and/or an adverse reaction associated with medical treatment in a subject, comprising molecular hydrogen as an active ingredient.

### RELATED APPLICATIONS

This application claims priority to Japanese Patent Application No. 2020-136852, filed on Jul. 21, 2020, the entire content of which is incorporated herein by reference.

### BACKGROUND OF THE INVENTION

### 1. Field of the Invention

The present invention provides a composition for prevention and/or improvement of an adverse drug reaction, a symptom associated with an adverse drug reaction, and/or an adverse reaction associated with medical treatment in subjects, comprising molecular hydrogen as an active ingredient.

### 2. Description of the Related Art

An adverse reaction refers to a secondary or undesirable effect of a drug or medical treatment. Any drug, including supplements, and any medical treatment that invades the body or any diagnostic procedure of a disease inevitably are associated with more or less adverse effects. Sometimes, such an adverse reaction may lead to a severe condition of the body.

Hydrogen, the active ingredient of the present invention, has anti-oxidative reactivity, which reduces oxidative stress caused by reactive oxygen species, and is known to have an improving effect against various diseases such as refractory cancer and a respiratory disease (Malcolm Dole, F. Ray Wilson, William P. Fife; Science, New Series, Vol. 190, No. 4210 (Oct. 10, 1975), pp. 152-154; Japanese Patent No. 6628449). MiZ Company Limited proposes a theory that hydrogen is the most effective means to selectively eliminate hydroxyl radicals produced in a mitochondrion because a hydrogen molecule can easily reach the inside of the mitochondrion. However, there is no precedent where the effect of use of hydrogen to prevent and/or improve an adverse drug reaction, a symptom associated with an adverse drug reaction, and/or an adverse reaction associated with medical treatment has been documented.

Prevention and/or improvement of an adverse drug reaction, a symptom associated with an adverse drug reaction, and/or an adverse reaction associated with medical treatment would make it possible to achieve relief of distress and improvement of quality of life for patients. As described above, however, few components or substances are known to be useful for prevention and/or improvement of an adverse drug reaction, a symptom associated with an adverse drug reaction, and/or an adverse reaction associated with medical treatment.

Under such circumstances, an object of the present invention is to prevent and/or improve an adverse drug reaction, a symptom associated with an adverse drug reaction, and/or an adverse reaction associated with medical treatment by using molecular hydrogen.

### SUMMARY OF THE INVENTION

That is, the present invention encompasses the following characteristics:

(1) A composition for prevention and/or improvement of an adverse drug reaction, a symptom associated with an adverse drug reaction, and/or an adverse reaction associated with medical treatment in a subject, comprising molecular hydrogen as an active ingredient.

(2) The composition according to (1), wherein the drug is a drug selected from the group consisting of anticancer agents, antihypertensive agents, antiparkinsonian drugs, antibiotics, antiallergic drugs, sleeping drugs, tranquilizers, antipyretic/analgesic/anti-inflammatory drugs, antibiotics, gastrointestinal drugs, vitamins, cold remedies, antiepileptic drugs, Chinese herbal medicines, liver disease treatment drugs, respiratory disease treatment drugs, cardiac disease treatment drugs, eye drops, erectile dysfunction treatment drugs, antidiabetic drugs, antifungal drugs, biologics, steroid drugs, antidementia drugs, muscular dystrophy treatment drugs, antipsychotic drugs, and anesthetic drugs.

(3) The composition according to (2), wherein the anticancer agent is a drug selected from the group consisting of alkylating agents (including ifosfamide, cyclophosphamide, dacarbazine, thiotepa, temozolomide, nimustine, busulfan, procarbazine, melphalan, ranimustine, carmustine, chlorambucil, and streptozocin), metabolic antagonists (including enocitabine, capecitabine, carmofur, cladribine, gemcitabine, cytarabine, cytarabine ocfosfate, tegafur, tegafur-uracil combination drugs, tegafur-gimeracil-oteracil potassium combination drugs, doxifluridine, hydroxycarbamide, fluorouracil, fludarabine, pemetrexed, pemetrexed sodium hydrates, methotrexate, mercaptopurine, clofarabine, nelarabine, and floxuridine), plant alkaloids (including irinotecan, etoposide, sobuzoxane, docetaxel, nogitecan, paclitaxel, vinorelbine, vincristine, vindesine, vinblastine, valrubicin, and liposomal daunorubicin), anticancer antibiotics (including actinomycin D, aclarubicin, amrubicin, idarubicin, epirubicin, zinostatin stimalamer, daunorubicin, doxorubicin, pirarubicin, bleomycin, peplomycin, mitomycin C, mitoxantrone, and liposomal doxorubicin), platinum agents (including oxaliplatin, carboplatin, cisplatin, and nedaplatin), hormonal agents (including anastrozole, exemestane, estramustine, ethinylestradiol, chlormadinone, goserelin, tamoxifen, dexamethasone, toremifene, bicalutamide, fadrozole, flutamide, prednisolone, fosfestrol, mitotane, methyltestosterone, medroxyprogesterone, mepitiostane, leuprorelin, letrozole, and fulvestrant), a biological response modulating agents (including interferons (α, β, and γ), interferons, ubenimex, Trametes versicolor polysaccharides, dried BCG, streptococcal extracts, and lentinan), molecular targeted drugs (including imatinib, gefitinib, gemtuzumab ozogamicin, tamibarotene, tretinoin, trastuzumab, bortezomib, rituximab, L-asparaginase, alemtuzumab, cetuximab, sunitinib, sorafenib, dasatinib, temsirolimus, and bevacizumab), immune checkpoint inhibitors including Opdivo; arsenic trioxide, calcium folinate, calcium levofolinate, docetaxel, sorafenib, erlotinib, crizotinib, gitecan (topotecan), vinorelbine, everolimus, goserelin, tamoxifen, emtansine, fulvestrant, prednisolone/methylprednisolone, lapatinib, octreotide, oxaliplatin, gimeracil, oteracil potassium, panitumumab, regorafenib, axitinib, teceleukin, temsirolimus, pazopanib, thalidomide, irinotecan, and endoxan as other anticancer agents; anticancer agents of classes of these agents; and derivatives thereof.

(4) The composition according to (2), wherein the antihypertensive drug is a drug selected from the group consisting of calcium antagonists, angiotensin converting enzyme inhibitors, angiotensin II receptor antagonists, diuretics, al blockers, blockers, central sympathetic nervous depressants (central a2 agonists), and derivatives thereof.

(5) The composition according to (2), wherein the antiparkinsonian agent is a drug selected from the group consisting of L-dopa, dopamine agonists, MAO-B inhibitors, catechol-O-methyltransferase inhibitors, amantadine (a dopamine release promoting drug), anticholinergic drugs, droxidopa (a noradrenaline supplement), zonisamide (a dopamine activating drug), adenosine receptor antagonists, and derivatives thereof.

(6) The composition according to (2), wherein the antibiotic is a drug selected from the group consisting of penicillin antibiotics, cephem antibiotics, macrolide antibiotics, tetracycline antibiotics, new quinolone antibiotics, antiviral drugs, antifungal drugs, antiprotozoal drugs, and derivatives thereof.

(7) The composition according to (2), wherein the antiallergic agent is a drug selected from the group consisting of mediator release inhibiting drugs, histamine H₁ receptor antagonists, thromboxane A₂ inhibitors, leukotriene LT receptor antagonists, Th2 cytokine inhibitors, and derivatives thereof.

(8) The composition according to (2), wherein the sleeping drug and the tranquilizer are drugs selected from the group consisting of nonbenzodiazepine sleeping drugs, benzodiazepines, melatonin receptor agonists, orexin receptor antagonists, barbiturates, and derivatives thereof.

(9) The composition according to (2), wherein the antipyretic/analgesic/anti-inflammatory drug is a disease selected from the group consisting of pyrine drugs, acetaminophen, nonsteroidal anti-inflammatory drugs, nonnarcotic analgesic drugs (opioid analgesics), and narcotic analgesic drugs.

(10) The composition according to (2), wherein the gastrointestinal drug is a drug selected from the group consisting of histamine Hi receptor antagonists, muscarine M₁ receptor antagonists, antacids, analgesic antispasmodic agents, and Chinese herbal medicines.

(11) The composition according to (2), wherein the vitamin is a drug selected from water-soluble vitamins and lipophilic vitamins.

(12) The composition according to (2), wherein the antiepileptic drug is a drug selected from the group consisting of hydantoin antiepileptic drugs, barbiturate antiepileptic drugs, triazine antiepileptic drugs, iminostil, and composite Aleviatin combination drugs.

(13) The composition according to (2), wherein the respiratory disease treatment drug is a drug selected from the group consisting of respiratory depression antagonists, respiratory center stimulating drugs, sleep apnea syndrome improving drugs, and respiratory distress syndrome improving drugs.

(14) The composition according to (2), wherein the liver disease treatment drug is a drug selected from the group consisting of hepatitis B treatment drugs, hepatitis C treatment drugs, autoimmune hepatitis treatment drugs, and liver cirrhosis treatment drugs.

(15) The composition according to (2), wherein the cardiac disease treatment drug is a drug selected from the group consisting of chronic heart failure treatment drugs; chronic heart failure treatment drugs selected from the group consisting of diuretics, cardiotonics, β blockers, and angiotensin II receptor antagonists/angiotensin converting enzyme inhibitors; ischemic heart disease treatment drugs selected from the group consisting of calcium antagonists, β blockers, nitric acid drugs, HMG-CoA reductase inhibitors (statins), antiplatelet drugs, and ARB/ACE inhibitors; and anticoagulants.

(16) The composition according to (2), wherein the antidiabetic drug is a drug selected from the group consisting of sulfonylureas, rapid-acting insulin secretion promoting drugs, DPP-4 inhibitors, biguanide drugs, thiazolidine drugs, α-glucosidase inhibitors, SLGT2 inhibitors, and combination drugs thereof.

(17) The composition according to (2), wherein the biologic is a drug selected from the group consisting of protein formulations, nucleic acid formulations, vaccines, blood formulations, antibody drugs, and similar biologics.

(18) The composition according to (2), wherein the antidementia drug is a drug selected from the group consisting of donepezil hydrochloride, galantamine, Exelon, rivastigmine, and memantine.

(19) The composition according to (2), wherein the anesthetic drug is a drug selected from the group consisting of isoflurane, desflurane, sevoflurane, xenon, nitrous oxide, thiopental sodium, thiamylal sodium, fentanyl, fentanyl citrate, remifentanil hydrochloride, droperidol/fentanyl citrate, ketamine hydrochloride, and propofol.

(20) The composition according to (1), wherein the medical treatment is treatment that invades the body, treatment in rehabilitation, or treatment involving electromagnetic radiation on the body.

(21) The composition according to (20), wherein the treatment that invades the body is one or more treatments selected from the group consisting of surgery, dialysis, blood transfusion, organ transplantation, cell transplantation, injection, and drip infusion.

(22) The composition according to (2), wherein the treatment involving electromagnetic radiation on the body is one or more treatments selected from the group consisting of x-ray radiography, MRI, CT, radiotherapy, or treatment using an infrared ray.

(23) The composition according to any one of (1) to (22), wherein the adverse drug reaction, the symptom associated with an adverse drug reaction, and/or the adverse reaction associated with medical treatment is one or more symptoms selected from the group consisting of sleepiness, dry throat, respiratory distress, pyrexia, itch on the body, rough skin, skin/mucous membrane erosion, blister, dry skin, body weight change, tinnitus, deafness, dermatitis, conjunctivitis, photosensitivity, nervousness, pigmentation, drug-induced hypersensitivity syndrome, chemical substance hypersensitivity, alopecia, hyperhidrosis, night sweats, dehydration symptoms, palpitations, shortness of breath, dyspnea, loss of appetite, insomnia, low back pain, dizziness, light-headedness, dizziness on standing up, hot flush, epilepsy, convulsion, numbness, coma, myocardial infarction, cerebral infarction, bronchial spasm, edema, dysgeusia, olfaction disorder, auditory abnormalities, burning sensation, cold sensation, chill, prickling sensation, tenderness, allodynia, seizure, coughing fit, hypoglycemia, hypoglycemic attack, mental confusion, disturbance of consciousness, memory impairment, dementia symptoms, excitation, delirium, visual impairment, malaise, fatigue, low mood, hallucination, nausea, vomiting, poor concentration, arrhythmia, abnormal electrocardiogram, abdominal pain, myalgia, muscle paralysis, muscle spasm, walking difficulty, headache, migraine, vascular pain, epigastralgia, injection site abnormalities, micturition pain, diarrhea, constipation, fibromyalgia, intestinal obstruction, exanthema, liver disorder, hepatic veno-occlusive disease, kidney disorder, renal tubular transport defect, hemorrhagic cystitis, pancreatitis, myelosuppression, hematopathy, multiple organ failure, asthma, pneumonia, pulmonary edema, pulmonary embolism, pulmonary fibrosis, jaundice, leukemia, osteomyelodysplasia, drug-induced interstitial pneumonia, arthralgia, diabetes mellitus, lymphadenopathy, calf cramps, hypokalemia, abnormal urine composition, uremia, lytic uremia, erythema, glaucoma, cataract, thrombocytopenia, leukopenia, leukocytosis, pancytopenia, agranulocytosis, infections, anemia, hypercalcemia, autoimmune hemolytic anemia, bacteremia, sepsis, heart failure, heart attack, cardiac asthma, atrioventricular block, urination disorder, rhabdomyolysis, infusion reaction, gastrointestinal ulcer, anaphylactic shock, Stevens-Johnson syndrome, inflammation, tonsillitis, stomatitis, glossitis, angular stomatitis, dry oral mucous membrane, gingival thickening, gingival hyperplasia, osteoporosis, bladder atrophy, hemiplegia, necrosis, erectile dysfunction, impotence, sexual debility, erectile impotence, testicular atrophy, aspermia, gynecomastia, menoxenia, amenorrhea, abnormal vaginal discharge, ovary fibrosis, immunodeficiency, tissue malformation, hemorrhage, hematuria, dysuria, melena, skin/nail atrophy, urticaria, adverse effect on the gonad, decreased blood pressure, elevated blood pressure, jaundice, asthmatic attack, normal cell injury, teratogenicity, drug-induced cancer, tumor lysis syndrome, angina pectoris, ascites, cerebral stroke, acute respiratory distress syndrome, DNA damage, worsened condition, depressive symptom, anxiety, and loss of QOL associated with these symptoms.

(24) The composition according to any one of (1) to (23), which is a liquid or a gas comprising the molecular hydrogen.

(25) The composition according to (24), wherein the liquid comprising the molecular hydrogen has a hydrogen concentration of 1 to 10 ppm.

(26) The composition according to (24), wherein the gas comprising the molecular hydrogen has a hydrogen concentration of higher than zero (0) and not higher than 18.5% by volume.

(27) The composition according to any one of (1) to (26), wherein the subject is a mammalian including a human.

(28) The composition according to any one of claims 1 to 27, which is produced by using a hydrogen gas generating apparatus, a hydrogen water generating apparatus, or a hydrogen gas adding apparatus.

The present invention can prevent and/or improve an adverse drug reaction, a symptom associated with an adverse drug reaction, and/or an adverse reaction associated with medical treatment in subjects.

### DETAILED DESCRIPTION

The present invention will be described in more detail below.
1. A Composition for Prevention and/or Improvement of an Adverse Drug Reaction, a Symptom Associated with an Adverse Drug Reaction, and/or an Adverse Reaction Associated with Medical Treatment

The present invention provides a composition for prevention and/or improvement of an adverse drug reaction, a symptom associated with an adverse drug reaction, and/or an adverse reaction associated with medical treatment, comprising molecular hydrogen as an active ingredient.

In the present specification, the term “adverse reaction” refers to a secondary or undesirable effect of drugs, food, supplements, or medical treatment.

In the present specification, the term “drug” includes, in addition to drugs and supplements, special-use food such as patient food and health food such as health-promoting food. Specific examples thereof include, but are not limited to, anticancer agents, antihypertensive agents, antiparkinsonian drugs, antibiotics, antiallergic drugs, sleeping drugs, tranquilizers, antipyretic/analgesic/anti-inflammatory drugs, antibiotics, gastrointestinal drugs, vitamins, cold remedies, antiepileptic drugs, Chinese herbal medicines, liver disease treatment drugs, respiratory disease treatment drugs, cardiac disease treatment drugs, eye drops, erectile dysfunction treatment drugs, antidiabetic drugs, antifungal drugs, biologics, steroid drugs, antidementia drugs, muscular dystrophy treatment drugs, antipsychotic drugs, and anesthetic drugs.

In the present specification, the term “subject” includes mammalians such as primates including humans, pet animals such as dogs and cats, and ornamental animals such as zoo animals. Preferred subjects are humans.

In the present specification, the term “anticancer agent” refers to, but is not limited to, an anticancer agent selected from the group consisting of alkylating agents (including ifosfamide, cyclophosphamide, dacarbazine, thiotepa, temozolomide, nimustine, busulfan, procarbazine, melphalan, ranimustine, carmustine, chlorambucil, and streptozocin), metabolic antagonists (including enocitabine, capecitabine, carmofur, cladribine, gemcitabine, cytarabine, cytarabine ocfosfate, tegafur, tegafur-uracil combination drugs, tegafur-gimeracil-oteracil potassium combination drugs, doxifluridine, hydroxycarbamide, fluorouracil, fludarabine, pemetrexed, pemetrexed sodium hydrate, methotrexate, mercaptopurine, clofarabine, nelarabine, and floxuridine), plant alkaloids (including irinotecan, etoposide, sobuzoxane, docetaxel, nogitecan, paclitaxel, vinorelbine, vincristine, vindesine, vinblastine, valrubicin, and liposomal daunorubicin), anticancer antibiotics (including actinomycin D, aclarubicin, amrubicin, idarubicin, epirubicin, zinostatin stimalamer, daunorubicin, doxorubicin, pirarubicin, bleomycin, peplomycin, mitomycin C, mitoxantrone, and liposomal doxorubicin), platinum agents (including oxaliplatin, carboplatin, cisplatin, nedaplatin), hormonal agents (including anastrozole, exemestane, estramustine, ethinylestradiol, chlormadinone, goserelin, tamoxifen, dexamethasone, toremifene, bicalutamide, fadrozole, flutamide, prednisolone, fosfestrol, mitotane, methyltestosterone, medroxyprogesterone, mepitiostane, leuprorelin, letrozole, and fulvestrant), biological response modulating agents (including interferons [α, β, and γ], interferons, ubenimex, Trametes versicolor polysaccharides, dried BCG, streptococcal extracts, and lentinan), molecular targeted drugs (including imatinib, gefitinib, gemtuzumab ozogamicin, tamibarotene, tretinoin, trastuzumab, bortezomib, rituximab, L-asparaginase, alemtuzumab, cetuximab, sunitinib, sorafenib, dasatinib, temsirolimus, and bevacizumab); immune checkpoint inhibitors including Opdivo; arsenic trioxide, calcium folinate, calcium levofolinate, docetaxel, sorafenib, erlotinib, crizotinib, gitecan (topotecan), vinorelbine, everolimus, goserelin, tamoxifen, emtansine, fulvestrant, prednisolone/methylprednisolone, lapatinib, octreotide, oxaliplatin, gimeracil, oteracil potassium, panitumumab, regorafenib, axitinib, teceleukin, temsirolimus, pazopanib, thalidomide, irinotecan, and endoxan as other anticancer agents; anticancer agents of classes of these agents; and derivatives thereof.

In the present specification, the term “antihypertensive drug” refers to, but is not limited to, an antihypertensive drug selected from the group consisting of calcium antagonists, angiotensin converting enzyme inhibitors, angiotensin II receptor antagonists, diuretics, al blockers, blockers, central sympathetic nervous depressants (central a2 agonists), and derivatives thereof.

In the present specification, the term “antiparkinsonian agent” refers to, but is not limited to, an antiparkinsonian agent selected from the group consisting of L-dopa, dopamine agonists, MAO-B inhibitors, catechol-O-methyl transferase inhibitors, amantadine (a dopamine release promoting drug), anticholinergic drugs, droxidopa (a noradrenaline supplement), zonisamide (a dopamine activating drug), adenosine receptor antagonists, and derivatives thereof.

In the present specification, the term “antibiotic” refers to, but is not limited to, an antibiotic selected from the group consisting of penicillin antibiotics, cephem antibiotics, macrolide antibiotics, tetracycline antibiotics, new quinolone antibiotics, antiviral drugs, antifungal drugs, antiprotozoal drugs, and derivatives thereof.

In the present specification, the term “antiallergic agent” refers to, but is not limited to, an antiallergic agent selected from the group consisting of mediator release inhibiting drugs, histamine H₁ receptor antagonists, thromboxane A₂ inhibitors, leukotriene LT receptor antagonists, Th2 cytokine inhibitors, and derivatives thereof.

In the present specification, the term “sleeping drug and tranquilizer” refers to a sleeping drug and a tranquilizer selected from the group consisting of nonbenzodiazepine sleeping drugs, benzodiazepines, melatonin receptor agonists, orexin receptor antagonists, barbiturates, and derivatives thereof.

In the present specification, the term “antipyretic/analgesic/anti-inflammatory drug” refers to, but is not limited to, an antipyretic/analgesic/anti-inflammatory drug selected from the group consisting of pyrine drugs, acetaminophen, nonsteroidal anti-inflammatory drugs, nonnarcotic analgesic drugs (opioid analgesics), and narcotic analgesic drugs.

In the present specification, the term “gastrointestinal drug” refers to, but is not limited to, a gastrointestinal drug selected from the group consisting of histamine H₁ receptor antagonists, muscarine Mi receptor antagonists, antacids, analgesic antispasmodic agents, and Chinese herbal medicines.

In the present specification, the term “vitamin” refers to, but is not limited to, a vitamin selected from water-soluble vitamins and lipophilic vitamins.

In the present specification, the term “antiepileptic drug” refers to, but is not limited to, an antiepileptic drug selected from the group consisting of hydantoin antiepileptic drugs, barbiturate antiepileptic drugs, triazine antiepileptic drugs, iminostil, and composite Aleviatin combination drugs.

In the present specification, the term “respiratory disease treatment drug” refers to, but is not limited to, a respiratory disease treatment drug selected from the group consisting of respiratory depression antagonists, respiratory center stimulating drugs, sleep apnea syndrome improving drugs, and respiratory distress syndrome improving drugs.

In the present specification, the term “liver disease treatment drug” refers to, but is not limited to, a liver disease treatment drug selected from the group consisting of hepatitis B treatment drugs, hepatitis C treatment drugs, autoimmune hepatitis treatment drugs, and liver cirrhosis treatment drugs.

In the present specification, the term “cardiac disease treatment drugs” refers to, but is not limited to, a cardiac disease treatment drug selected from the group consisting of chronic heart failure treatment drugs; chronic heart failure treatment drugs selected from the group consisting of diuretics, cardiotonics, β blockers, and angiotensin II receptor antagonists/angiotensin converting enzyme inhibitors; ischemic heart disease treatment drugs selected from the group consisting of calcium antagonists, β blockers, nitric acid drugs, HMG-CoA reductase inhibitors (statins), antiplatelet drugs, and ARB/ACE inhibitors; and anticoagulants.

In the present specification, the term “antidiabetic drug” refers to, but is not limited to, an antidiabetic drug selected from the group consisting of sulfonylureas, rapid-acting insulin secretion promoting drugs, DPP-4 inhibitors, biguanide drugs, thiazolidine drugs, α-glucosidase inhibitors, SLGT2 inhibitors, and combination drugs thereof.

In the present specification, the term “biologic” refers to, but is not limited to, a biologic selected from the group consisting of protein formulations, nucleic acid formulations, vaccines, blood formulations, antibody drugs, and similar biologics.

In the present specification, the term “anesthetic drug” refers to, but is not limited to, an anesthetic drug selected from the group consisting of isoflurane, desflurane, sevoflurane, xenon, nitrous oxide, thiopental sodium, thiamylal sodium, fentanyl, fentanyl citrate, remifentanil hydrochloride, droperidol/fentanyl citrate, ketamine hydrochloride, and propofol.

In the present specification, the term “medical treatment” refers to an action of treating/diagnosing/caring/rehabilitating a disease of a human, an animal, or the like. Specifically, the term refers to, but is not limited to, treatment that invades the body, treatment in rehabilitation, treatment involving electromagnetic radiation on the body, or the like.

In the present specification, the term “treatment that invades the body” refers to, but is not limited to, treatment selected from the group consisting of surgery, dialysis, blood transfusion, organ transplantation, cell transplantation, injection, and drip infusion.

In the present specification, the term “treatment involving electromagnetic radiation on the body” refers to, but is not limited to, x-ray radiography, MRI, CT, radiotherapy, or treatment using an infrared ray.

In the present specification, “dialysis” refers to, but is not limited to, hemodialysis or peritoneal dialysis.

In the present specification, “an adverse drug reaction, a symptom associated with the adverse drug reaction, and/or an adverse reaction associated with the medical treatment” refers to, but is not limited to, one or more symptoms selected from the group consisting of sleepiness, dry throat, respiratory distress, pyrexia, itch on the body, rough skin, skin/mucous membrane erosion, blister, dry skin, body weight change, tinnitus, deafness, dermatitis, conjunctivitis, photosensitivity, nervousness, pigmentation, drug-induced hypersensitivity syndrome, chemical substance hypersensitivity, alopecia, hyperhidrosis, night sweats, dehydration symptoms, palpitations, shortness of breath, dyspnea, loss of appetite, insomnia, low back pain, dizziness, light-headedness, dizziness on standing up, hot flush, epilepsy, convulsion, numbness, coma, myocardial infarction, cerebral infarction, bronchial spasm, edema, dysgeusia, olfaction disorder, auditory abnormalities, burning sensation, cold sensation, chill, prickling sensation, tenderness, allodynia, seizure, coughing fit, hypoglycemia, hypoglycemic attack, mental confusion, disturbance of consciousness, memory impairment, dementia symptoms, excitation, delirium, visual impairment, malaise, fatigue, low mood, hallucination, nausea, vomiting, poor concentration, arrhythmia, abnormal electrocardiogram, abdominal pain, myalgia, muscle paralysis, muscle spasm, walking difficulty, headache, migraine, vascular pain, epigastralgia, injection site abnormalities, micturition pain, diarrhea, constipation, fibromyalgia, intestinal obstruction, exanthema, liver disorder, hepatic veno-occlusive disease, kidney disorder, renal tubular transport defect, hemorrhagic cystitis, pancreatitis, myelosuppression, hematopathy, multiple organ failure, asthma, pneumonia, pulmonary edema, pulmonary embolism, pulmonary fibrosis, jaundice, leukemia, osteomyelodysplasia, drug-induced interstitial pneumonia, arthralgia, diabetes mellitus, lymphadenopathy, calf cramps, hypokalemia, abnormal urine composition, uremia, lytic uremia, erythema, glaucoma, cataract, thrombocytopenia, leukopenia, leukocytosis, pancytopenia, agranulocytosis, infections, anemia, hypercalcemia, autoimmune hemolytic anemia, bacteremia, sepsis, heart failure, heart attack, cardiac asthma, atrioventricular block, urination disorder, rhabdomyolysis, infusion reaction, gastrointestinal ulcer, anaphylactic shock, Stevens-Johnson syndrome, inflammation, tonsillitis, stomatitis, glossitis, angular stomatitis, dry oral mucous membrane, gingival thickening, gingival hyperplasia, osteoporosis, bladder atrophy, hemiplegia, necrosis, erectile dysfunction, impotence, sexual debility, erectile impotence, testicular atrophy, aspermia, gynecomastia, menoxenia, amenorrhea, abnormal vaginal discharge, ovary fibrosis, immunodeficiency, tissue malformation, hemorrhage, hematuria, dysuria, melena, skin/nail atrophy, urticaria, adverse effect on the gonad, decreased blood pressure, elevated blood pressure, jaundice, asthmatic attack, normal cell injury, teratogenicity, drug-induced cancer, tumor lysis syndrome, angina pectoris, ascites, cerebral stroke, acute respiratory distress syndrome, DNA damage, worsened condition, depressive symptom, anxiety, and loss of QOL associated with these symptoms.

In the present specification, “hydrogen,” the active ingredient of the composition of the present invention, is molecular hydrogen (i.e., gaseous hydrogen or hydrogen gas) and is simply referred to as “hydrogen” or “hydrogen gas” unless otherwise specified. Additionally, the term “hydrogen” used in the present specification refers to a molecular formula of H₂, D₂ (deuterium), or HD (deuterated hydrogen) or a gas mixture thereof. D₂ is expensive but known to have a stronger superoxide eliminating effect than that of H₂. Hydrogen that can be used in the present invention is H₂, D₂ (deuterium), HD (deuterated hydrogen), or a gas mixture thereof, preferably H₂. Alternatively, D₂, and/or HD can be used instead of H₂ or in a mixture with H₂.

Preferred embodiments of the composition of the present invention are gases or liquids containing molecular hydrogen, preferably gases containing molecular hydrogen.

The gases containing molecular hydrogen are preferably air containing hydrogen gas or a mixed gas containing hydrogen gas and oxygen gas. The concentration of hydrogen gas in a gas containing molecular hydrogen (i.e., the composition of the present invention) is higher than zero (0) and not higher than 18.5% by volume, for example, 0.5% to 18.5% by volume, preferably 1% to 10% by volume, for example, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, and the like. In the present invention, higher hydrogen gas concentrations (but below the explosion limit) or higher daily hydrogen doses tend to be associated with greater effects of promoting prevention and/or improvement (e.g., suppression or alleviation) of an adverse drug reaction, a symptom associated with an adverse drug reaction, and/or an adverse reaction associated with medical treatment.

Because hydrogen is a flammable and explosive gas, it is preferable to add hydrogen to the composition of the present invention under conditions safe for subjects such as humans and administer the mixture to subjects to improve an adverse drug reaction, a symptom associated with an adverse drug reaction, and/or an adverse reaction associated with medical treatment.

When a gas other than hydrogen gas is air, the air concentration is in the range of, for example, 81.5% to 99.5% by volume.

When a gas other than hydrogen gas is a gas containing oxygen gas, the oxygen gas concentration is in the rage of, for example, 21% to 99.5% by volume.

As another main gas, for example, nitrogen gas can be further added.

In a usual hydrogen gas inhalation therapy, an effect of improving a disease (cancer) is observed only when a hydrogen gas is used at a high concentration of 66% or 99%. In the present invention, however, it is preferable to add hydrogen to the composition of the present invention under safe conditions for subjects such as humans and administer it to a subject, and a sufficient effect of improving an adverse drug reaction, a symptom associated with an adverse drug reaction, and/or an adverse reaction associated with medical treatment can be exhibited even at low hydrogen concentrations of higher than 0 (zero) and 18.5% or lower.

The liquids containing molecular hydrogen are specifically aqueous liquids containing a dissolved hydrogen gas. Examples of the aqueous liquids used herein include, but are not limited to, water (e.g., purified water, sterilized water), physiological saline, buffer solutions (e.g., buffer solutions of pH 4 to 7.4), drip infusion solutions, fluid infusion solutions, injection solutions, and drinks (e.g., tea drinks such as green tea and black tea, fruit juice, green juice, vegetable juice). Examples of the hydrogen concentration in a liquid containing molecular hydrogen include, but are not limited to, 1 to 10 ppm, preferably 1.2 to 9 ppm, for example, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, and 5 to 7 ppm.

In the present invention, higher concentrations of dissolved hydrogen (but below the explosion limit) or higher daily hydrogen doses tend to be associated with greater effects of preventing and/or improving an adverse drug reaction, a symptom associated with an adverse drug reaction, and/or an adverse reaction associated with medical treatment.

A gas or a liquid containing molecular hydrogen is formulated to provide a predetermined hydrogen gas concentration and then with the same, for example, a pressure-resistant container (e.g., a stainless cylinder, an aluminum can, a pressure-resistant plastic bottle [e.g., a pressure-resistant PET bottle] and a plastic bag preferably having the inside laminated with an aluminum film, or an aluminum bag) is filled. Aluminum has the property of unlikely allowing hydrogen molecules to pass therethrough. Alternatively, a gas containing molecular hydrogen or a liquid containing molecular hydrogen may be produced in situ before use by using an apparatus such as a hydrogen gas generating apparatus, a hydrogen water generating apparatus, or a hydrogen gas adding apparatus such as a known or commercially available hydrogen gas supply apparatus (an apparatus for generating a gas containing molecular hydrogen), a hydrogen adding device (an apparatus for hydrogen water generation), or a non-destructive hydrogen adding apparatus (e.g., an apparatus for non-destructively adding hydrogen gas into a bag for a biocompatible solution such as a drip infusion solution).

The hydrogen gas supply apparatus enables hydrogen gas generated from a reaction of a hydrogen generating agent (e.g., metallic aluminum, magnesium hydride) and water to be mixed with a diluent gas (e.g., air, oxygen) in a predetermined ratio (refer to Japanese Patent No. 5228142, etc.). Or, the hydrogen gas supply apparatus mixes hydrogen gas generated utilizing electrolysis of water with a diluent gas such as oxygen or air (refer to Japanese Patent No. 5502973, Japanese Patent No. 5900688, etc.). Thus, a gas containing molecular hydrogen at a hydrogen concentration in the range of, for example, 0.5% to 18.5% by volume can be prepared.

The hydrogen adding device is an apparatus that generates hydrogen by using a hydrogen generating agent and a pH modifier and dissolving the hydrogen in a biocompatible solution such as water (refer to Japanese Patent No. 4756102, Japanese Patent No. 4652479, Japanese Patent No. 4950352, Japanese Patent No. 6159462, Japanese Patent No. 6170605, Japanese Patent Laid-open No. 2017-104842, etc.). Examples of a mixture of a hydrogen generating agent and a pH modifier include metallic magnesium and a strongly acidic ion exchange resin or an organic acid (e.g., malic acid, citric acid) and a metallic aluminum powder and a calcium hydroxide powder. With these mixtures, a liquid containing molecular hydrogen at a dissolved hydrogen concentration of, for example, approximately 1 to 10 ppm can be prepared.

The non-destructive hydrogen adding apparatus is an apparatus or a device that adds hydrogen gas to a commercially available biocompatible solution such as a drip infusion solution (e.g., enclosed in a hydrogen-permeable plastic bag such as a polyethylene bag) from the outside of a package and is commercially available from, for example, MiZ Company Limited (http://www.e-miz.co.jp/technology.html). This apparatus can dissolve hydrogen in a biocompatible solution aseptically until the equilibrium concentration is reached, by immersing a bag containing the biocompatible solution in saturated hydrogen water, so that hydrogen is permeated into the bag. The apparatus is composed of, for example, an electrolytic bath and a water bath, and water in the water bath is circulated in the electrolytic bath and the water bath to generate hydrogen by electrolysis. Or, a simplified, disposable device can be used for a similar purpose (refer to Japanese Patent Laid-open No. 2016-112562, etc.). This device has a biocompatible solution-containing plastic bag (a hydrogen-permeable bag, for example, a polyethylene bag) and a hydrogen generating agent (e.g., metallic calcium, metallic magnesium/cation exchange resin) incorporated in an aluminum bag, and the hydrogen generating agent is wrapped with, for example, a non-woven fabric (e.g., steam-permeable non-woven fabric). Hydrogen generated by wetting the hydrogen generating agent wrapped with a non-woven fabric with a small amount of water, such as a steam, is dissolved in a biocompatible solution non-destructively and aseptically.

Or, a purified hydrogen gas cylinder, a purified oxygen gas cylinder, or a purified air cylinder may be provided to produce a gas or a liquid containing molecular hydrogen which is adjusted to provide a predetermined hydrogen concentration or a predetermined oxygen or air concentration.

A gas containing molecular hydrogen or a liquid containing molecular hydrogen (e.g., water [e.g., purified water, sterilized water], physiological saline, drip infusion solution) prepared using the above-mentioned apparatuses or devices can be administered orally or parenterally to subjects.

Other embodiments of the composition of the present invention include dosage forms (e.g., tablets, capsules) prepared to be orally administered to (or ingested by) subjects, which contain a hydrogen generating agent that enables hydrogen to be generated in the gastrointestinal tract. The hydrogen generating agent preferably comprises, for example, components approved as food or food additives.

When the composition of the present invention comprises molecular hydrogen as an active ingredient, examples of the method of administering the composition to subjects include administration by inhalation, suction or the like. For example, transpulmonary administration is preferred. When a liquid containing molecular hydrogen is contained as an active ingredient, oral or intravenous administration (including drip infusion) is preferred. When a gas is inhaled, the gas is inhaled from the mouth or the nose via a nasal cannula or a mask-like device covering the mouth and the nose, transported to the lungs, and delivered to the whole body by blood.

The liquid containing molecular hydrogen to be orally administered may be administered to subjects as a cooled liquid or a liquid stored at room temperature. Hydrogen is dissolved in water at a concentration of approximately 1.6 ppm (1.6 mg/L) at room temperature and under a normal pressure, and the difference in solubility due to temperature is known to be relatively small. Or, when a liquid containing molecular hydrogen is, for example, in the form of a drip infusion solution or an injection solution containing hydrogen gas prepared using the above-described non-destructive hydrogen adding apparatus, the liquid may be administered to subjects by parenteral routes, such as intravenous or intraarterial administration.

One dose or multiple doses (e.g., two to three doses) per day of a gas containing molecular hydrogen at the above-mentioned hydrogen concentrations or a liquid containing molecular hydrogen at the above-mentioned dissolved hydrogen concentrations can be administered to humans for a period of one week to three months or longer, for example, one week to six months or longer (e.g., one year or longer, two years or longer). When a gas containing molecular hydrogen is administered, the gas is preferably inhaled for at least 30 minutes per dose. Because the improving effect becomes higher with a longer inhalation time, the gas can be administered for, for example, 30 minutes to one hour, one hour to two hours, two hours to three hours, or longer. Additionally, when a gas containing molecular hydrogen is administered in a transpulmonary manner by inhalation or suction, the gas can be administered to subjects under an atmospheric pressure environment, or, for example, under a high atmospheric pressure in the range exceeding a standard atmospheric pressure (i.e., approximately 1.013 atm) and not higher than 7.0 atm, for example, under a high atmospheric pressure environment in the range of 1.02 to 7.0 atm, preferably in the range of 1.02 to 5.0 atm, more preferably in the range of 1.02 to 4.0 atm, yet more preferably in the range of 1.02 to 1.35 atm (including the gas containing molecular hydrogen).
2. A method for preventing and/or improving an adverse drug reaction, a symptom associated with an adverse drug reaction, and/or an adverse reaction associated with medical treatment

The composition containing molecular hydrogen, an adverse drug reaction, a symptom associated with an adverse drug reaction, and/or an adverse reaction associated with medical treatment, dose, administration method, and the like are as described in the above 1.

In the method of the present invention, a gas containing molecular hydrogen (preferably, air or oxygen) at higher than zero (0) and not higher than 18.5% by volume, for example, 0.5% to 18.5% by volume, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, or the like, preferably 5% to 10% by volume, 5% to 8% by volume, for example, 6% to 10% by volume, 6% to 8% by volume, 6% to 7% by volume, or the like can be inhaled or sucked by subjects for, for example, one to three hours or longer per day and can be continued for, for example, one to three months or longer, four to seven months or longer, one to three years or longer.

Or, in the method of the present invention, for example, 200 to 500 mL per dose for intravenous administration or, for example, 500 to 1000 mL per dose for oral administration of a liquid containing molecular hydrogen at a concentration of, for example, 1 to 10 ppm, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, 5 to 7 ppm, or the like, preferably 3 to 10 ppm, 4 to 10 ppm, 5 to 10 ppm, 5 to 9 ppm, 5 to 8 ppm, 5 to 7 ppm, or the like can continue to be administered to subjects for, for example, 0.5 to three months or longer, four to seven months or longer, one to three years or longer.

The method of the present invention comprises inhalation by a subject for at least 30 minutes or longer, one hour or longer, two hours or longer, three hours or longer, four hours or longer, five hours or longer, or six hours or longer per day.

In the method of the present invention, the amount of a hydrogen gas inhaled by a subject using a hydrogen gas supply apparatus or a cylinder may be greater than 0 (zero) mL/min and 5 mL/min or smaller, 10 mL/min or smaller, 20 mL/min or smaller, 30 mL/min or smaller, 40 mL/min or smaller, 50 mL/min or smaller, 100 mL/min or smaller, 200 mL/min or smaller, 300 mL/min or smaller, 400 mL/min or smaller, 500 mL/min or smaller, 1000 mL/min or smaller, 1200 mL/min or smaller, 1500 mL/min or smaller, or 2000 mL/min or smaller.

### EXAMPLE

The present invention is explained more specifically with reference to the following example. However, the example is not intended to limit the scope of the present invention.

### Example 1

### <Case 1: A Case of Improvement of Adverse Drug Reactions to Anticancer Agents by Hydrogen Inhalation>

A 38-year-old female patient was diagnosed with undifferentiated soft tissue sarcoma in blood vessels of the heart and the lungs and underwent surgery to remove sarcoma in the heart, but sarcoma metastasized from the heart to the lungs, where blood vessels gather, could not be removed. She continued to receive treatment with anticancer agents (eight cycles) and treatment with Opdivo in parallel and experienced adverse drug reactions of lost hair on the head and rough skin. In parallel with these treatments, the patient initiated hydrogen gas suction using a hydrogen gas generator MHG2000a (manufactured by MiZ Company Limited: a hydrogen gas concentration, 6.6% by volume; hydrogen, approximately 120 mL/min), which prepares a mixture gas of hydrogen and air. Approximately one month later, the rough skin started improving, and the skin started becoming resilient. Following the treatment with anticancer agents, she continued hydrogen gas inhalation for six months. Then, the hair on the head, which usually requires time to start growing, started growing.

### <Case 2: A Case of Improvement of Adverse Drug Reactions to Anticancer Agents by Hydrogen Inhalation>

A 68-year-old female patient with an unknown primary cancer was receiving treatment with anticancer agents to treat cancer metastasized to the rib bone and suffering from adverse drug reactions of malaise and rough skin. Then, in parallel with these treatments, the patient started suction of a hydrogen gas using a hydrogen gas generator Jobs-a (manufactured by MiZ Company Limited: a hydrogen gas concentration 4% to 5% by volume; approximately 200 mL/min), which prepares a mixture gas of hydrogen and air, and drinking 7 ppm hydrogen water. She inhaled a hydrogen gas for three to five hours per day and drank 500 mL of hydrogen water per day. After she continued to inhale a hydrogen gas and drink hydrogen water for three months, the skin started becoming lustrous, and malaise subsided.

### <Case 3: A Case of Improvement of Adverse Drug Reactions to Anticancer Agents by Hydrogen Inhalation>

A 45-year-old male patient with esophageal cancer experienced an adverse drug reaction of persistent insomnia due to treatment with anticancer agents, lost appetite because of malaise, and could not even go out. He initiated hydrogen gas inhalation using a hydrogen gas generator Jobs-mini (manufactured by MiZ Company Limited: a hydrogen gas concentration 1.3% by volume; approximately 30 mL/min). The inhalation time per day was two to three hours. Following the second hydrogen gas inhalation, he felt warmth in the head probably because of the improved blood circulation, could sleep soundly, and woke up with a good feeling. Then, the quality of sleep improved, the body felt light, and the patient was able to go out. When he drank too much alcohol, he used to have a severe hangover. However, when he inhaled hydrogen before going to sleep, a hangover started improving earlier than usual.

### <A Case of Improvement of an Adverse Drug Reaction to an Antibacterial Agent by Hydrogen Inhalation>

A 70-year-old man had a common cold and took a new quinolone antibacterial agent. He lost strength in the body and was taken to hospital by ambulance. The physician made a diagnosis of rhabdomyolysis caused by the antibacterial agent. He was discharged from hospital one week later, but the tiredness persisted, and he had difficulty in putting strength into the limbs. Because people around him suggested him, he started suction of a hydrogen gas using a hydrogen gas generator MHG2000a (manufactured by MiZ Company Limited: a hydrogen gas concentration, 6.6% by volume; hydrogen, approximately 120 mL/min). After he continued hydrogen gas inhalation (90 minutes per day) for two weeks, he became gradually able to put strength into the limbs and experienced reduced tiredness. His condition improved to the extent that he could have a walk. The patient is sure that the improvement is due to the effect of hydrogen to improve the adverse drug reaction.

### <A Case of Improvement of Adverse Drug Reactions to Antiallergic Drugs by Hydrogen Inhalation>

A 45-year-old male patient with cedar pollinosis had been taking antiallergic agents (an antihistamine, a nasal drop, and an eye drop) prescribed by a physician in March every year, but he suffered adverse drug reactions of sleepiness and tiredness after taking drugs. The patient started inhaling a hydrogen gas (for 90 minutes to three hours per day) using a hydrogen gas generator MHG2000a (manufactured by MiZ Company Limited: a hydrogen gas concentration, 6.6% by volume; hydrogen, approximately 120 mL/min) and drinking 7 ppm hydrogen water (500 mL to 1 L per day). Three days later, the tiredness of the body felt after taking the drugs were reduced, and he felt that the head was cleared.

### <A Case of Improvement of Adverse Reactions to Hemodialysis by Hydrogen Inhalation>

A 65-year-old female patient developed renal failure as a complication of diabetes mellitus and has been receiving hemodialysis since 2006. Although she had used more than 20 and less than 30 different drugs, she experienced no symptom improvement and also had a symptom of walking difficulty.

Therefore, she inhaled a hydrogen gas using a hydrogen generator (device model, Jobs-a manufactured by MiZ Company Limited [a hydrogen concentration, approximately 5%; other gas, air]; an amount of hydrogen gas generated, 200 mL/min), with an inhalation time of three to four hours per day.

The patient was able to walk from 10 days after the initiation of hydrogen inhalation. Additionally, edema was reduced, and she felt better from one month after the initiation of hydrogen inhalation although she usually had developed edema and felt sluggish after receiving dialysis. A clear improving effect of hydrogen inhalation was observed.

### <A Case of Improvement of Adverse Reactions to Radiotherapy by Hydrogen Inhalation>

A male patient with cancer received radiotherapy and felt malaise and nausea after the therapy. When he came home, he initiated hydrogen gas suction using a hydrogen gas generator MHG2000 (manufactured by MiZ Company Limited: a hydrogen gas concentration, 3% to 4% by volume; hydrogen, approximately 120 mL/min), on the suggestion of his family. At one hour after the start of inhalation, he felt the body becoming light. Nausea was gradually resolving 90 minutes later. Therefore, the patient continues hydrogen gas inhalation after radiotherapy to reduce adverse reactions.

The present invention can prevent and/or improve an adverse drug reaction, a symptom associated with an adverse drug reaction, and/or an adverse reaction associated with medical treatment by administering a composition containing molecular hydrogen.

What is claimed is:

1. A method for preventing and/or improving an adverse drug reaction, a symptom associated with an adverse drug reaction, and/or an adverse reaction associated with medical treatment in a subject, comprising administering to the subject an effective amount of molecular hydrogen.

2. The method according to claim 1, wherein the drug is a drug selected from the group consisting of anticancer agents, antihypertensive agents, antiparkinsonian drugs, antibiotics, antiallergic drugs, sleeping drugs, tranquilizers, antipyretic/analgesic/anti-inflammatory drugs, antibiotics, gastrointestinal drugs, vitamins, cold remedies, antiepileptic drugs, Chinese herbal medicines, liver disease treatment drugs, respiratory disease treatment drugs, cardiac disease treatment drugs, eye drops, erectile dysfunction treatment drugs, antidiabetic drugs, antifungal drugs, biologics, steroid drugs, antidementia drugs, muscular dystrophy treatment drugs, antipsychotic drugs, and anesthetic drugs.

3. The method according to claim 1, wherein the medical treatment is a treatment that invades the body, a treatment in rehabilitation, or a treatment involving electromagnetic radiation on the body.

4. The method according to claim 3, wherein the treatment that invades the body is one or more treatments selected from the group consisting of surgery, dialysis, blood transfusion, organ transplantation, cell transplantation, injection, and drip infusion.

5. The method according to claim 4, wherein the treatment involving electromagnetic radiation on the body is one or more treatments selected from the group consisting of x-ray radiography, MRI, CT, radiotherapy, and treatment using an infrared ray.

6. The method according to claim 1, wherein the adverse drug reaction, the symptom associated with an adverse drug reaction, and/or the adverse reaction associated with medical treatment is one or more symptoms selected from the group consisting of sleepiness, dry throat, respiratory distress, pyrexia, itch on the body, rough skin, skin/mucous membrane erosion, blister, dry skin, body weight change, tinnitus, deafness, dermatitis, conjunctivitis, photosensitivity, nervousness, pigmentation, drug-induced hypersensitivity syndrome, chemical substance hypersensitivity, alopecia, hyperhidrosis, night sweats, dehydration symptoms, palpitations, shortness of breath, dyspnea, loss of appetite, insomnia, low back pain, dizziness, light-headedness, dizziness on standing up, hot flush, epilepsy, convulsion, numbness, coma, myocardial infarction, cerebral infarction, bronchial spasm, edema, dysgeusia, olfaction disorder, auditory abnormalities, burning sensation, cold sensation, chill, prickling sensation, tenderness, allodynia, seizure, coughing fit, hypoglycemia, hypoglycemic attack, mental confusion, disturbance of consciousness, memory impairment, dementia symptoms, excitation, delirium, visual impairment, malaise, fatigue, low mood, hallucination, nausea, vomiting, poor concentration, arrhythmia, abnormal electrocardiogram, abdominal pain, myalgia, muscle paralysis, muscle spasm, walking difficulty, headache, migraine, vascular pain, epigastralgia, injection site abnormalities, micturition pain, diarrhea, constipation, fibromyalgia, intestinal obstruction, exanthema, liver disorder, hepatic veno-occlusive disease, kidney disorder, renal tubular transport defect, hemorrhagic cystitis, pancreatitis, myelosuppression, hematopathy, multiple organ failure, asthma, pneumonia, pulmonary edema, pulmonary embolism, pulmonary fibrosis, jaundice, leukemia, osteomyelodysplasia, drug-induced interstitial pneumonia, arthralgia, diabetes mellitus, lymphadenopathy, calf cramps, hypokalemia, abnormal urine composition, uremia, lytic uremia, erythema, glaucoma, cataract, thrombocytopenia, leukopenia, leukocytosis, pancytopenia, agranulocytosis, infections, anemia, hypercalcemia, autoimmune hemolytic anemia, bacteremia, sepsis, heart failure, heart attack, cardiac asthma, atrioventricular block, urination disorder, rhabdomyolysis, infusion reaction, gastrointestinal ulcer, anaphylactic shock, Stevens-Johnson syndrome, inflammation, tonsillitis, stomatitis, glossitis, angular stomatitis, dry oral mucous membrane, gingival thickening, gingival hyperplasia, osteoporosis, bladder atrophy, hemiplegia, necrosis, erectile dysfunction, impotence, sexual debility, erectile impotence, testicular atrophy, aspermia, gynecomastia, menoxenia, amenorrhea, abnormal vaginal discharge, ovary fibrosis, immunodeficiency, tissue malformation, hemorrhage, hematuria, dysuria, melena, skin/nail atrophy, urticaria, adverse effect on the gonad, decreased blood pressure, elevated blood pressure, jaundice, asthmatic attack, normal cell injury, teratogenicity, drug-induced cancer, tumor lysis syndrome, angina pectoris, ascites, cerebral stroke, acute respiratory distress syndrome, DNA damage, worsened condition, depressive symptom, anxiety, and loss of QOL associated with these symptoms.

7. The method according to claim 1, wherein the molecular hydrogen is administered as a liquid composition or a gas composition comprising the molecular hydrogen.

8. The method according to claim 7, wherein the liquid composition has a hydrogen concentration of 1 to 10 ppm.

9. The method according to claim 7, wherein the gas composition has a hydrogen concentration of higher than zero (0) and not higher than 18.5% by volume.

10. The method according to claim 1, wherein the subject is a mammal.

11. The method according to claim 1, wherein the subject is a human.

12. The method according to claim 7, wherein the liquid or gas composition is produced by using a hydrogen gas generating apparatus, a hydrogen water generating apparatus, or a hydrogen gas adding apparatus.
